(19) United States
(12) Patent Application Publication
(10) Pub. No.: US 2019/0385730 A1
(43) Pub. Date: Dec. 19, 2019

(54) System and Method for Separately Managing Product Information and Customer Preferences and for Dispensing a Product from a Point of Sale Vending Machine

(71) Applicants: Steven Franklin Baugh, Broomfield, CO (US); Peter de Boves Harrington, Athens, OH (US)

(72) Inventors: Steven Franklin Baugh, Broomfield, CO (US); Peter de Boves Harrington, Athens, OH (US)

(21) Appl. No.: 15/998,044

(22) Filed: Jun. 16, 2018

(51) Int. Cl. G16H 20/90; G16H 20/10; G16H 50/30; G16H 10/60; G06Q 30/06

(52) U.S. Cl. CPC G16H 20/90; G16H 20/10; G06Q 30/0631; G16H 10/60; G16H 50/30

(57) ABSTRACT

A method and system for optimizing product selection based on chemical attribute declustering. The system receives a product selection from a user, receives preference feedback data from the user including at least one physical attribute or medicinal effect, such as personal feelings or a calming effect for the product selection, and processes the preference feedback data, which may include a geometric location, to generate a three-dimensional representation including indicia of the preference feedback data to assist the user in product selection. The three-dimensional representation has at least one area correlating to the physical or medicinal effects. The product selection can be a food product selection, an herbal product, a drug product, or a cannabis product. A vending machine can be provided to formulate a chemical composition from a number of ingredients which are blended immediately for distribution to a patient or customer.

### FIELD OF THE INVENTION

The present application relates to systems and methods for managing product information and customer preferences and, more particularly, to a system and method for separately managing objective product information and subjective customer preferences and for dispensing a product from a point of sale vending machine.

### BACKGROUND OF THE INVENTION

In the past, patients and consumers have typically been directed to certain foods, lifestyles, exercise programs, essential oils and/or herbal medicines based on other's experiences and recommendations, rather than evidence-based approaches or personalized medicine.

Botanical preparations, nutrition, and traditional medicines are based upon thousands of years of experience and empirical data. However, the complex relationships between the ingredients are not necessarily explained in Western medicine terms. The ingredients, relative amounts, and processing are controlled to continue the tradition of healing. Recently, Western medicine has taken historically effective complex mixtures and has reduced them to single compound medicines while the sought-after effects are diluted over larger patient or customer populations. These concentrated, single compound drugs are evaluated in large clinical trials, the larger the better, in which the effects of individual differences are reduced to an average value that is then applied to the population.

As interest in personalized medicine and individual variation in treatment effects, prescription drug interactions, complex nutritional requirements, exercise, and alternative medicines increases, managing the data in an intuitive and useful manner gets increasingly difficult for the physician and patient alike. None of the compounds or relationships exists in isolation, and in complex drug discovery and personal health management they are potentially all related and affect each other, necessitating complex statistical models to correlate the data. To this end, analyzing combustion products from a smoke stream, for example, has become more important in recent years.

It has become evident that medication genetics, per se, has not always been a foolproof or even a good predictor of outcome for every patient. Depending on the chemistry of the patients themselves, a given strain and dosage of a medication may result in significant variances of results from one patient to the next. Therefore, to guarantee a certain medication will result in favorable results for a particular patient, the patient's chemistry must be analyzed and considered prior to prescribing his medication and dosage. This step in treating the patient can be more important than merely knowing the genetics of the medication.

The education necessary to understand the powerful models takes the data out of the hands of the very consumer, patient, physician, or personal trainer in need of the data. Conveying the information needed to allow the individual or health care professional to make decisions based on the ever-increasing amount of data gets exponentially more difficult. As more and more researchers and laboratories identify health relationships related to diet, exercise, or herbal products, the customer is less and less able to process and use this information. The Complex Drug Discovery model is an intuitive graphical representation of data that enables the customer to manage his health and overall wellbeing by making decisions based on data that can be managed by the customer/patient or in consultation with his physician.

While statistics have been applied to many complex problems, the person directing and performing the data interpretation had to be an expert in statistics, often programming his own complex algorithms and data pretreatment. Note: for purposes of this description, the pronouns, “he” and “his” are used to refer to antecedents of indeterminate gender, implicitly and explicitly including both men and women.

Data pretreatment is often used when a desired outcome or correlation is already observed and the data need to demonstrate this agreement. What is needed, then, is a method whereby the original data are all preserved in the data universe, and these data are not influenced by individual responses or preferences.

The music streaming service, Pandora®, operated by Pandora Media, Inc., is an example of a mathematically driven preference system. Pandora starts with a brief sketch of what the customer already likes, and then suggests more artists that the customer might like. These algorithms incorporate similar results of other people's questionnaires into consideration when recommending new music he might like.

Another similar technology often used to collect objective data about products is near infrared (nIR) spectral analysis. In the current art, samples of ingredients are analyzed to build a spectral library of each ingredient. New lots of incoming ingredients are then compared to the spectral library for consistency. Samples outside the specification are rejected. While nIR may provide a certain measure of quality control, it fails to address the provision of substitute or alternative components that, while not identical, provide either the same or even better objective or subjective experience for the customer. Moreover, as a tool to reverse engineer the combustion products, it has limited usefulness.

### Description of Related Art

U.S. Pat. No. 8,676,937 issued to Rapaport, et al. for SOCIAL-TOPICAL ADAPTIVE NETWORKING (STAN) SYSTEM ALLOWING FOR GROUP BASED CONTEXTUAL TRANSACTION OFFERS AND ACCEPTANCES AND HOT TOPIC WATCHDOGGING on Mar. 18, 2014 describes a Social-Topical Adaptive Networking (STAN) system that can inform users of cross-correlations between currently focused-upon topic or other nodes in a corresponding topic or other data-objects organizing space maintained by the system and various social entities monitored by the system. More specifically, one of the cross-correlations may be as between the top N now-hottest topics being focused-upon by a first social entity and the amounts of focus “heat” that other social entities (e.g., friends and family) are casting on the same topics or other subregions of other cognitive attention receiving spaces in a relevant time period.

U.S. Pat. No. 8,274,377 issued to Smith, et al. for INFORMATION COLLECTING AND DECISION MAKING VIA TIERED INFORMATION NETWORK SYSTEMS on Sep. 25, 2012 describes techniques, apparatus and systems for information collecting and decision making based on one or more tiered networks of sensors and communication nodes for security monitoring and warning, disaster warning, counter-terrorism, and other applications associated with information collecting and decision making.

U.S. Published Patent Application No. 2007/0115475 on application by Shpantzer for SYSTEM AND METHOD FOR CHEMICAL SENSING USING TRACE GAS DETECTION published on May 24, 2007 describes a system and method for chemicals detection such as explosives and others, which are based on sensing of trace gases associated with the chemical. This sensing includes detection of spectrum and relative concentration of the trace gases followed by the chemical identification based on these data.

What is needed is a system whereby the customer can take recommendations from a database. Alternatively, sampling strategies can be developed to cover the available inventory quickly and efficiently. Opposite corners of a tetrahedron can quickly generate geometric relationships across the three-dimensional space, and then the patient takes his own experiences into account and directs his own sampling versus being given recommendations based on others. This is important when considering personalized medicine and biochemical individuality. For example, someone seeking chemotherapy will not have any previous experience or preference.

### SUMMARY OF THE INVENTION

The present system and method enables a 3D representation of all the data without the myriad details that lie behind the sample results and determining its 3D position in the data universe. The customer can then intuitively select the “dots” that represent specific products within the graphical representation of the inventory to experiment with. The customer/patient reaction or preference to those products is tracked in his own graphical subset of the entire universe and subjective and objective data are not confounded. The graphs are initially generated using a common ingredient list for each product in a specific category, such as the ingredients from three or more chai tea recipes, and are then subjected to multivariate analysis and/or otherwise can generate graphical representations of data, such as with the products MATLAB® manufactured by MathWorks, Inc. or Solo™ manufactured by Eigenvector Research, Incorporated.

The ingredients of each product may be provided by manufacturers or chemical compositions can be generated using one or more well understood chemical analysis applications or tools such as those in the field of chromatography or spectral analysis. In sum, a product profile or ingredient list is provided from pre-existing sources and is then stored in a database for use by the present system.

Once the customer/patient decides on his preference, his preferences are collected via one or more input devices, such as a mobile phone, computer, or kiosk, and stored in a “personal data universe” as well as a general indication/reaction database. In this way, only selected preferences are admitted to the general indication/reaction database and grouped accordingly.

This preference data can be harvested any number of ways, including recommending products to new customers/patients, finding market trends, and developing new products to fill blank spaces in the chemical composition data universe that are indicated by trends or lack of product choices in a given space. Moreover, effective products can be compared to similar but non-effective medications to arrive at meaningful differences, versus myriad differences presented by a distant, non-efficacious product.

A vending machine can be provided to formulate a chemical composition from a number of ingredients which are blended immediately for distribution to a patient or customer.

### BRIEF DESCRIPTION OF THE DRAWINGS

A complete understanding of the present invention may be obtained by reference to the accompanying drawings, when considered in conjunction with the subsequent detailed description, in which:

FIG. 1 shows a spider plot of the chemical profile of an essential oil generated using a software program that can perform multivariable analysis;

FIG. 2 shows an alternative plot of the recipe profile of an essential oil using a two-dimensional graphical representation as an output from the multivariable analysis software;

FIG. 3 shows a second alternative plot of the chemical profile of an essential oil using a two-dimensional graphical representation;

FIG. 4 is a graphical representation of 30 selected ingredients that comprise 18 different chai tea recipes, the graphical representation being de-clustered to allow maximum resolution of the recipes;

FIG. 5 illustrates a graph representing outcomes of a first selection of three products by a customer in response to interaction with a graphical representation of each product in accordance with the present invention;

FIG. 6 illustrates one sample graph that can be provided to a customer using color in the graphical representation to illustrate one or more components that may be recommended for trial use by the current system;

FIG. 7 illustrates a sample graph illustrating the step of highlighting one or more recommended selections in conjunction with earlier trials and personal experience data;

FIG. 8 illustrates a graphical interface for enabling a customer to navigate the available inventory by combining the first and second results of the sampling data to identify a more optimal product choice;

FIG. 9 provides a graphical representation of a sample that the present system might recommend for a customer;

FIG. 10 illustrates a sample graphic representation of selected favorite or preferred product based on product composition;

FIG. 10A is a flowchart illustrating optimization of personalized product development with the consumer or patient at the center of the process;

FIG. 11 provides a diagram illustrating the data components of the system of the present invention;

FIG. 12 is a graphical representation of one sample geometric location in a recommended product formulation;

FIG. 13 a flowchart illustrating the method for enabling a customer to create an account and utilize the present invention;

FIG. 14 is a flowchart illustrating a method for integrating the customer feedback and utilization to identify potentially new or desired products;

FIG. 15 is a graph showing a sativa indica hybrid chemical profile that unsuccessfully attempts to correlate to strain;

FIG. 16 is a graph showing a successful correlation of chemical profile to outcome

FIG. 17 is a schematic view of a chemical profile cube such as can be viewed on a vending machine to geometrically blend five ingredients to form two products; and

FIG. 18 is another schematic view of a chemical profile cube to geometrically blend seven ingredients to form two products.

Like reference numerals refer to like parts throughout the several views of the drawings.

### DETAILED DESCRIPTION OF EMBODIMENTS

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

One or more different inventions may be described in the present application. Further, for one or more of the invention(s) described herein, numerous embodiments are described in this patent application for illustrative purposes only.

The described embodiments are not intended to be limiting in any sense. One or more of the inventions may be widely applicable to numerous embodiments, as is readily apparent from the disclosure. These embodiments are described in sufficient detail to enable those skilled in the art to practice one or more of the inventions, and it is to be understood that other embodiments may be utilized and that structural, logical, software, electrical and other changes may be made without departing from the scope of the one or more of the inventions. Accordingly, those skilled in the art will recognize that the one or more of the inventions may be practiced with various modifications and alterations.

Particular features of one or more of the inventions may be described with reference to one or more particular embodiments or figures that form a part of the present disclosure, and in which are shown, by way of illustration, specific embodiments of one or more of the inventions. It should be understood, however, that such features are not limited to usage in the one or more particular embodiments or figures with reference to which they are described. The present disclosure is neither a literal description of all embodiments of one or more of the inventions nor a listing of features of one or more of the inventions that must be present in all embodiments.

A description of an embodiment with several components in concert with each other does not imply that all such components are required. To the contrary, a variety of optional components are described to illustrate the wide variety of possible embodiments of one or more of the inventions.

Moreover, the illustration of a process by its depiction in a drawing does not imply that the illustrated process is exclusive of other variations and modifications thereto, does not imply that the illustrated process or any of its steps are necessary to one or more of the inventions, and does not imply that the illustrated process is preferred.

When a single device or article is described, it will be readily apparent that more than one device/article (whether or not they cooperate) may be used in place of a single device/article. Similarly, where more than one device or article is described (whether or not they cooperate), it will be readily apparent that a single device/article may be used in place of the more than one device or article.

The functionality and/or the features of a device may be alternatively embodied by one or more other devices that are not explicitly described as having such functionality/features. Thus, other embodiments of one or more of the inventions need not include the device itself.

The invention is a method and system for optimizing product selection based on chemical attribute de-clustering. The system receives a product selection from a user, receives preference feedback data from the user including at least one physical attribute or medicinal effect, such as personal feelings or a calming effect for the product selection, and processes the preference feedback data, which may include a geometric location, to generate a three-dimensional representation including indicia of the preference feedback data to assist the user in product selection.

This system and method differ from similar products in several pivotal ways. First, other methods, not unlike a soda flavoring machine, understand only what is being sold, but do not interact with the customer to know if he is merely experimenting or the present selection is his favorite. Other methods using previous reference-based recommendations do not span all the macro groups and make recommendations across groups or genres like music. For example, the Pandora music streaming service provides recommendations within the country music genre, but does not consider classical or pop or hip hop. Music programs identify the user's preferences and recommend new songs based on those preferences by comparison to others with similar preferences. Pandora also does not have a song writing team and band to create new songs or a new musical genre once it identifies and projects trends or identifies gaps in the musical universe. Nor does Pandora allow a person to pick his own songs from the inventory graphically. They are all recommended to the user.

In contrast, by providing a customer means for documenting so called subjective or preference data, the current system and method provides a better understanding of not just whether he prefers it, but how much he prefers it and why he prefers it.

The current system maintains multiple stocks or supplies with specific chemical profiles to substitute or recommend new vendors for a customer when his original suppliers are not available or useable or to identify new available products that may fit his profile. As a result, if a manufacturer stops making a product, a substitution must be made. The present system and method uses spectral data to identify a material from another manufacturer that meets the current customer specifications and subjective input that could be used to optimize this substitution. Currently, if a product is dropped, the purchaser's library is discarded and a new vendor is identified, followed by a new spectral library. If the supplier maintained these records, then rejected lots for one customer could be identified for use by another customer, and substitutions could be provided.

Additionally, product characteristics could be identified and new products developed to fill market opportunities identified statistically as voids in the inventory data universe. However, as the principal components used in spectral methods like nIR are not directly attributable or traceable to product characteristics or chemical composition, unique compositions are difficult to predict and relationships to specific chemicals are not established in complex mixtures for drug discovery. Current industry practices use spectral data, in which a product preference can be identified, but the differences between the different samples are not anchored in chemical profiles. These differences are manifested only in meaningless product or strain names, and chemical profiles are then needed for all samples, if still available, to elucidate differences, making strains and cultures a diversion, not an answer for complex drug discovery.

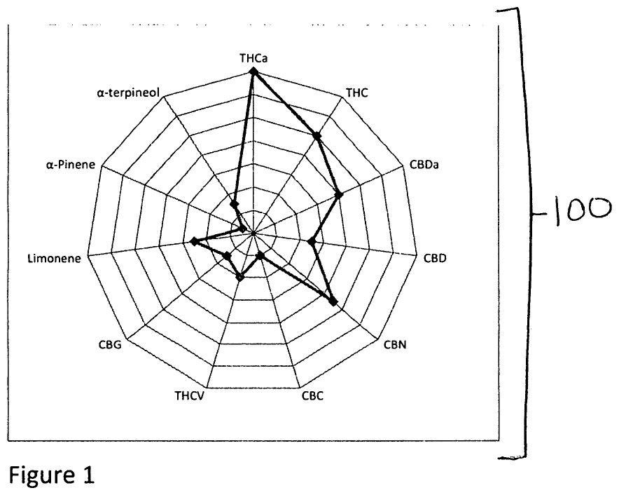

As an initial matter, to understand the unique method and system of the present invention, it is important to understand the unique graphical representation and data sets that can be used to implement the present invention. For example, referring now to FIG. 1, a sample spider plot 100 of the chemical profile of an essential oil is shown. The axes are standardized and only the shape is necessary moving forward. The two-dimensional representation reduces the need for the legends, thereby reducing the distractions and helping the customer or any user to visualize the data. The present invention uses these 2D graphical representations to correlate data regarding the chemical composition with one or more customer preferences as explained hereinbelow.

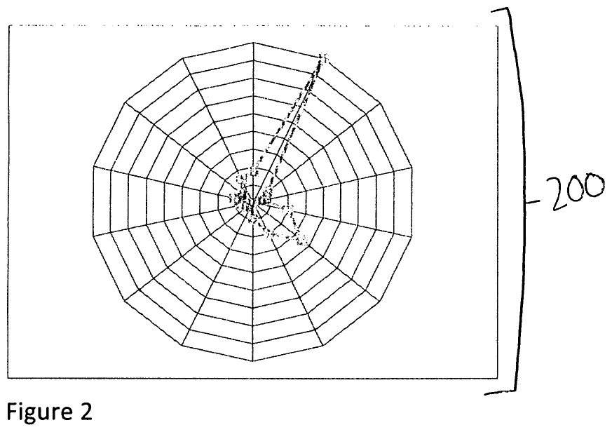

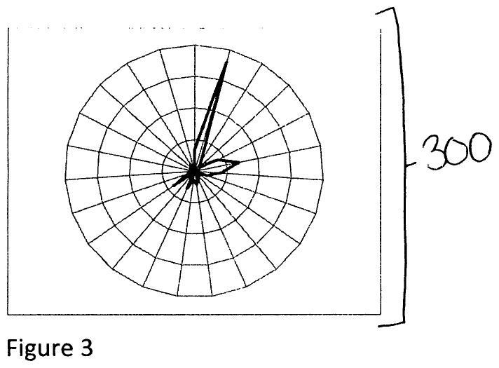

FIGS. 2 and 3 illustrate alternative plots 200, 300 of the chemical profile of an essential oil using a two-dimensional graphical representation. In accordance with the present invention, these sample two-dimensional plots can be useful as customers experience one or more products in a category, such as chai tea, and want to use the present system and method to compare specific compounds, classes of compounds, or other limited variables.

For example, a plot graph similar to the plot graphs 200, 300 can be used to correlate weight loss and other factors against the chemical composition of a sample product to identify which components are most strongly correlated with weight loss or weight optimization.

Similar graphs can be generated using data entered by a customer into the system such as personal lifestyle questionnaires retrieved from one or more product providers, or nutritional value scores from participating product companies. The information in databases, such as included in the Oxygen Radical Absorbance Capacity (ORAC) database on fruits and vegetables, and other specific researchers or industries, can also be leveraged to identify consumer and marketing trends.

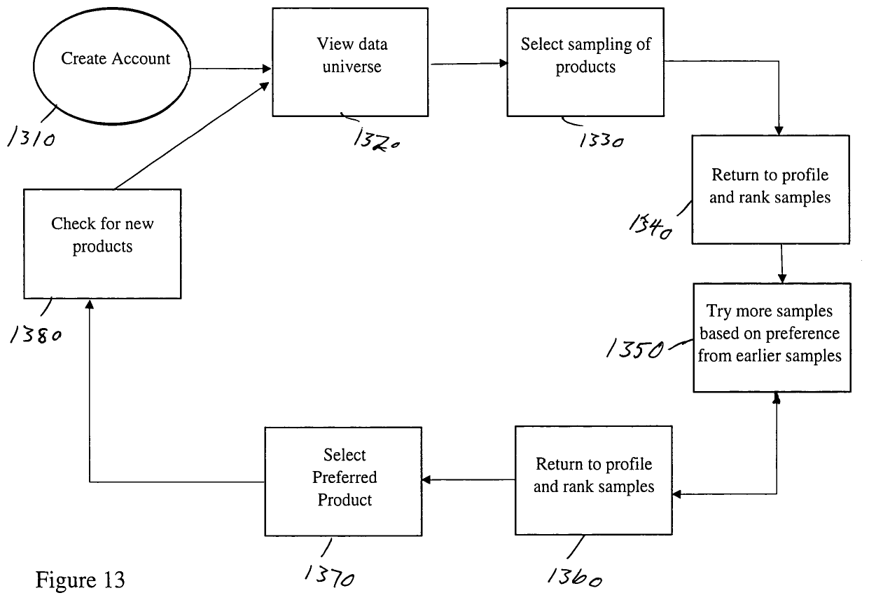

As further described with reference to FIG. 13, a customer can select which preferences to track and save in his profile based on what is meaningful to him based on his personal preferences or based on input from a physician or personal trainer. These preferences, in conjunction with the components in the product, can be fed into programs that can perform the multivariable analysis, such as MATLAB® or Solo™ to assess the relationship between his personal preferences and the components of the product.

As mentioned, the two-dimensional representations 200, 300 do not have axes that are labeled, to increase the ease of visualization. The representation 200, 300 may or may not have labeled axes based on information density, and the customer can choose the graphing options and save the profile in his emerging medical history.

Multivariate statistics can be used to cluster products having similar chemical compositions, but the present invention provides multiple graphical representations by illustrating differences between different “views” of the data that may also illustrate seeming small differences between the product and the consumer experience with the product to de-cluster the results and illustrate product differences.

An example of such a process would be a machine learning application that identifies a knife, fork, and spoon all as “eating utensils.” That grouping is a generalization and removes the differences between the items. The present system can provide one or more graphical representations to illustrate differences by de-clustering the inventory and magnifying differences in line with the customer's chemical data or personal preference data or whatever view that he may desire. By de-clustering the inventory, the present system provides the customer with more choices by revealing small differences between products/personal preferences that can be important based on one or more of the profile data entered by the customer that correlates the chemical composition of the product with those preferences in accordance with customer selection and input.

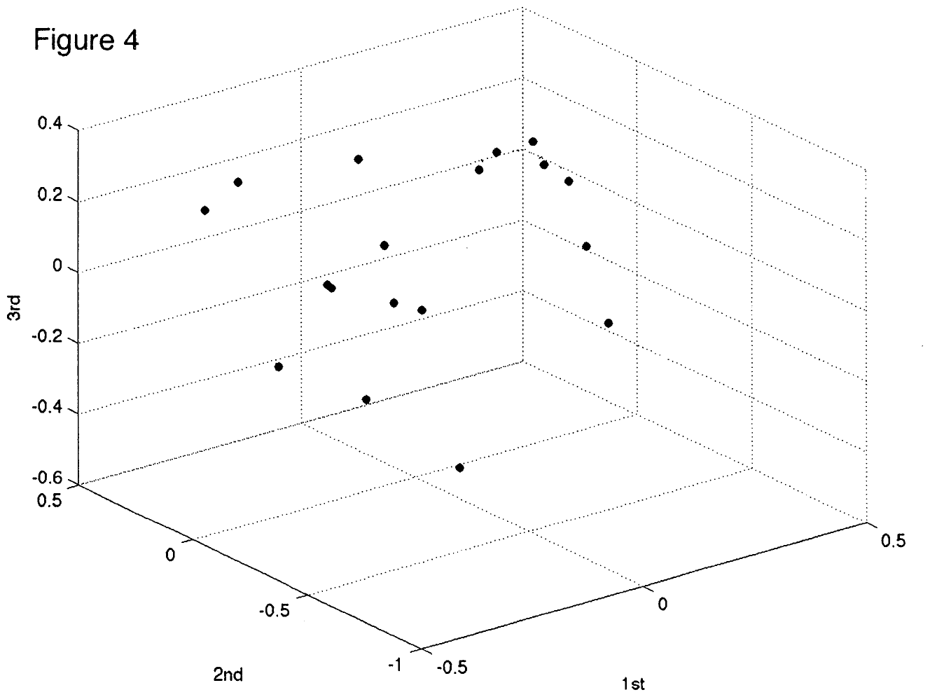

FIGS. 4 through 10 illustrate graphical representations 400, 500, 600, 700, 800, 900, 1000, respectively, that can be generated by the current system to help guide a customer to selecting a preferred chai tea recipe. The graphs illustrate one method for providing a visual illustration of 18 different chai tea recipes that have been de-clustered using approximately 30 ingredients with normalized amounts.

Referring now to FIG. 4, there is shown a graphical representation 400 of the chai tea ingredients data, de-clustered to allow maximum resolution of the available recipes without regard to the personal data of the customer. The present invention allows this ongoing manipulation of data collected during the personalization phase in an intuitive, geometrical model, thereby permitting an intuitive visual model that can help a consumer better identify consumer products and services that have greater efficacy.

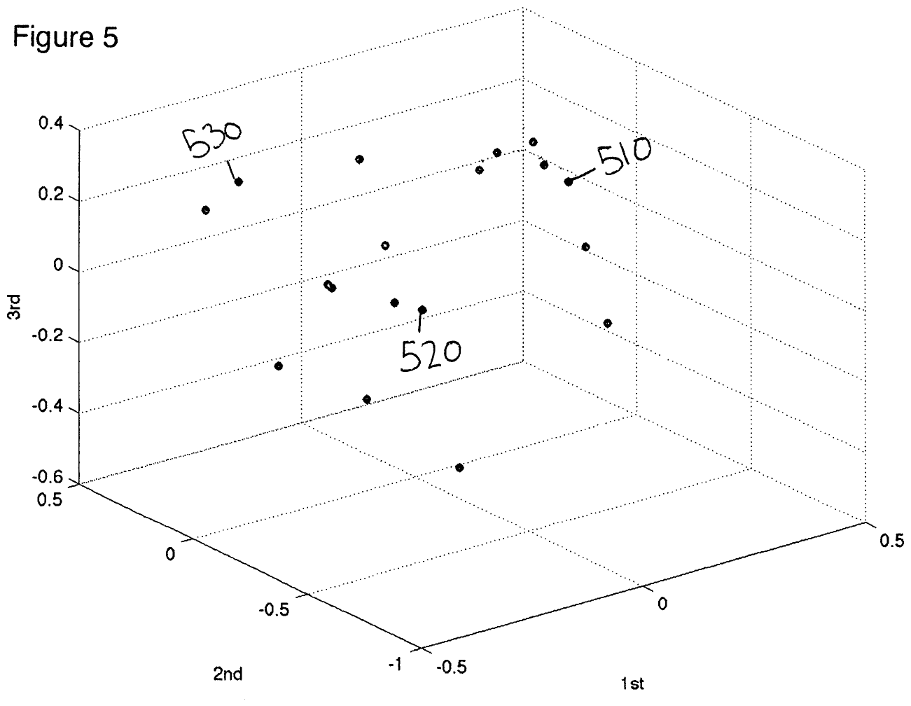

FIG. 5 provides an illustration demonstrating a first selection of three samples 510, 520, 530 and represents a sample outcome of a customer making three different chai tea choices. In the illustrated embodiment, these first three choices are highlighted in black, and are designed to cover the maximum area of the available choices by selecting the broadest range of distance between each of the sample choices in accordance with the analysis.

In the preferred embodiment, the customer or patient would take or consume the selected three samples 510, 520, 530 for evaluation. The customer then returns to the system to enter his personal experiences. For example, the customer could enter a number, such as a range from 1 to 10, or using a color scheme or using another way of communicating quantitative data graphically, to input his preferences into the system by way of a device—such as a kiosk, mobile device, computer or other common computing device (not shown). In the following example three simple colors are used: red for dislike, yellow for neutral and green for like.

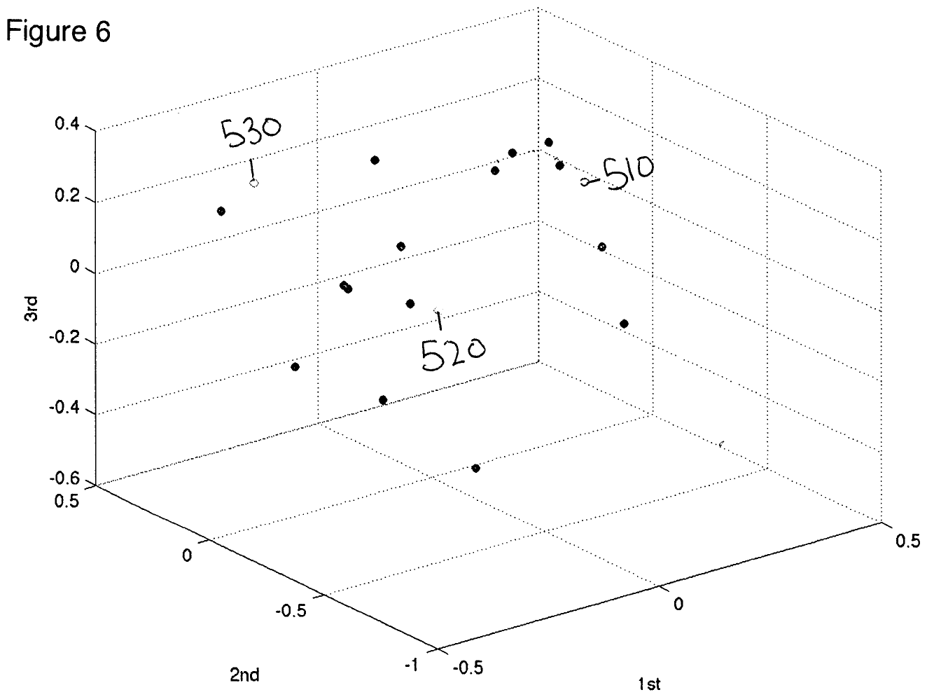

FIG. 6 provides an illustration of how the results could be entered into the system visually by permitting the user to change the colors of the dots representing the samples 510, 520, 530 that are being reviewed by the user.

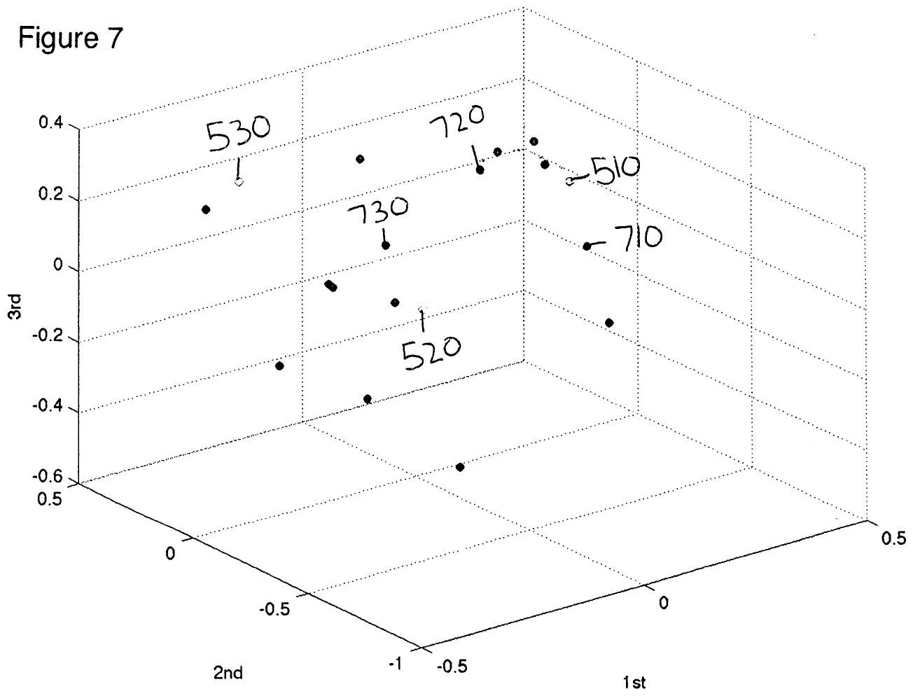

FIG. 7 shows the next three selections 710, 720, 730 highlighted in black while retaining the earlier results for reference. Using the feedback from the first three samples, a new sampling strategy is developed. This process can be intuitive on the part of the patient, or performed by a physician or expert/consultant in the case of specialized products, wine, or cigars. The system then uses these inputs to recommend three new samples, 710, 720, 730 that are selected around the area of the positive results, while reaching into adjacent areas of the product inventory.

Following the customer trying selections 710, 720, 730, the customer returns to enter his results into the system. The colors on the selected sample dots 710, 720, 730 are changed to the color of his review. As can be seen, a pattern of results will develop. Certain customers will be against spicy chai while others will prefer it, for example. This could be the case with the upper left quadrant of the chai example. In the second round of sampling, this area is avoided in favor of more choices around favorable reviews. In this way the customer will navigate the available inventory while closing in on his favorite.

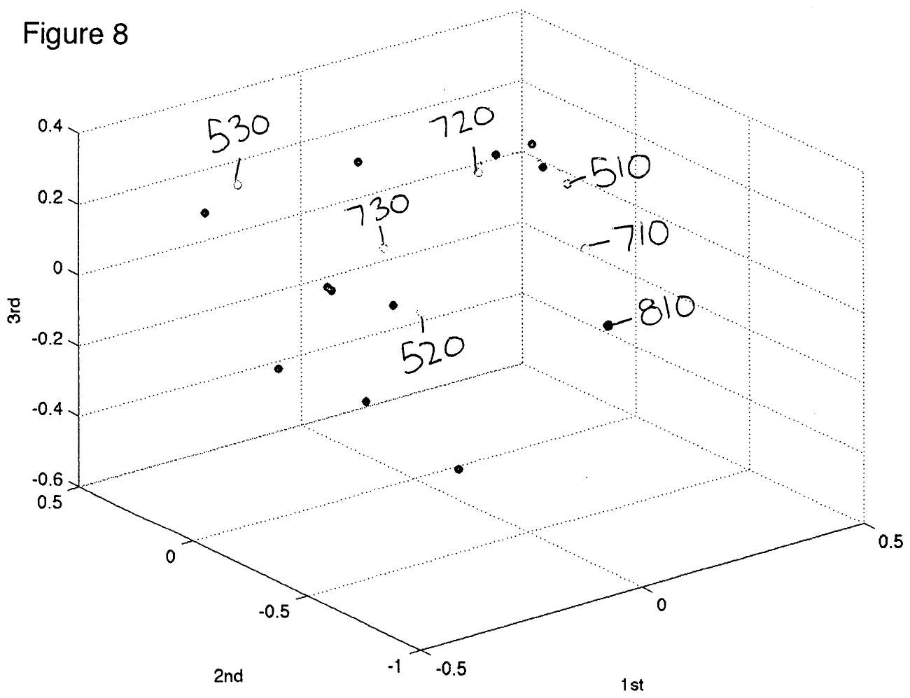

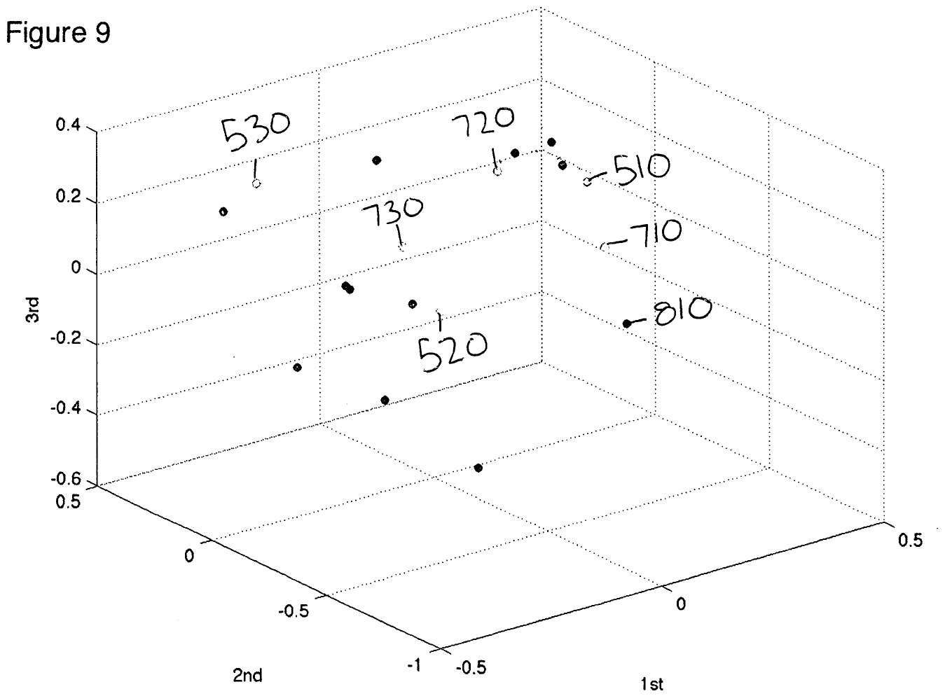

FIG. 8 shows the theoretical second set of results 710, 720, 730 combined with the first 520, 520, 530 to provide a graphical representation of the input customer preferences in relations to the products tested. As can be seen by the graphical representation in FIG. 8, the customer's indicated preferences demonstrate increased desire for products that include components down and to the right. In this limited data set there is only one sample 910 remaining to move towards. The customer selects a sample of that product in FIG. 9 and takes it home for evaluation. In this case it would be useful to create additional recipes for chai using ingredients and amounts that could fill in the voids around the customer's new favorite as the graphical representation shows that there are fewer products available in the portion of the representation that appear to correlate with the customer's inputted preferences.

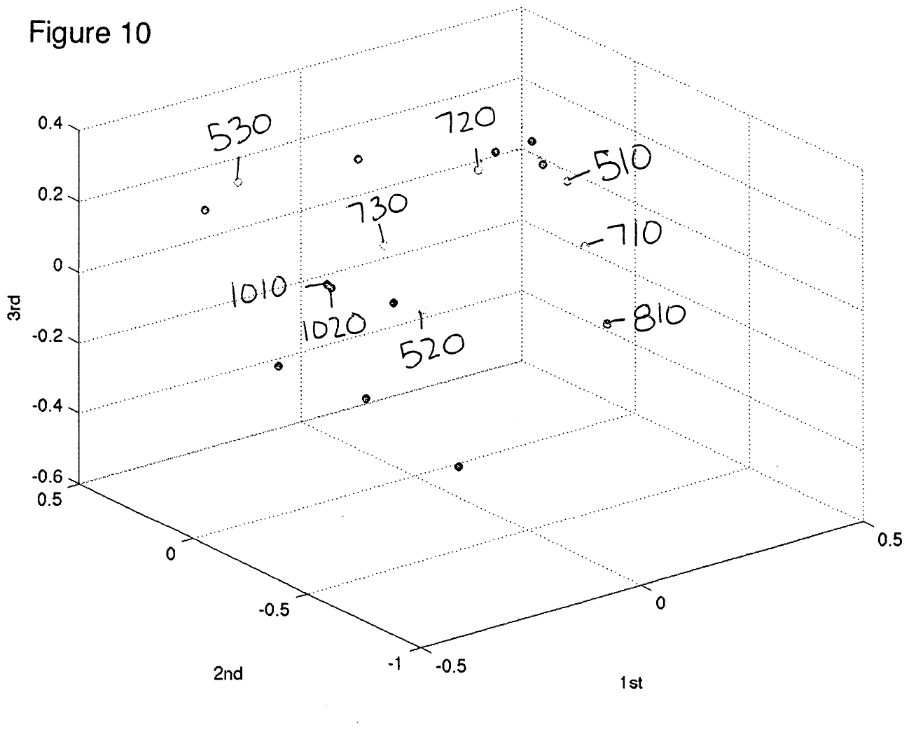

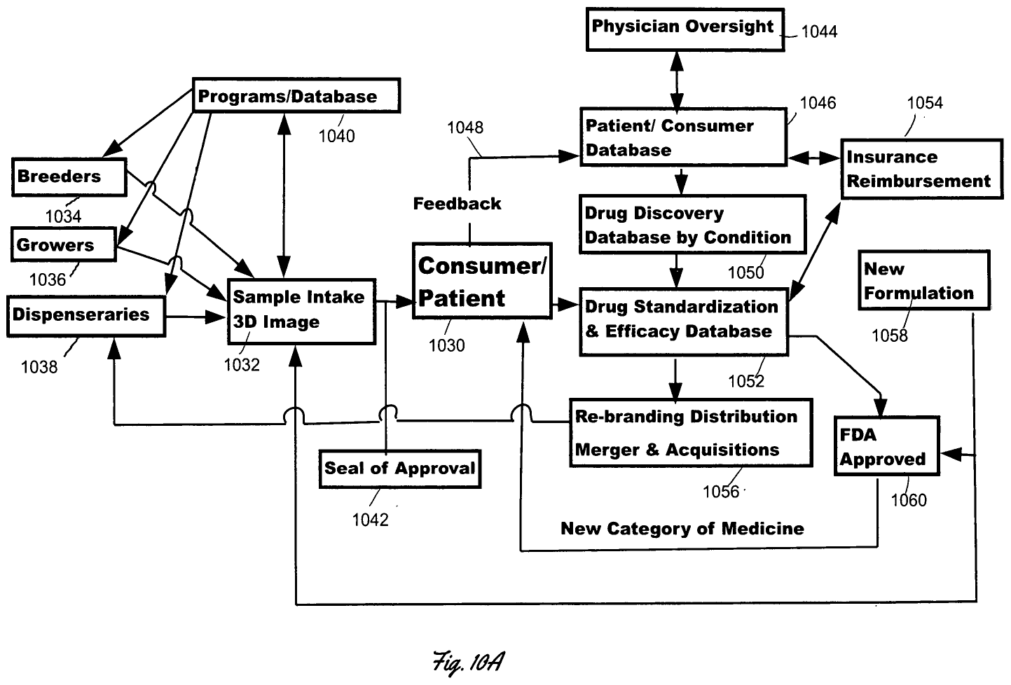

FIG. 10 demonstrates a sample graphical representation following the customer selection of his new favorite. In particular, the now purple dot 810 corresponds to the product that the customer selected as his new favorite. However, there are only a few other choices in that area of the three-dimensional space. Information such as this could be provided to product development companies to help shape the development of more products if customers continue to select that sample as their favorite in that portion of the graphical space. This data can also be used in the classical sense in that it is obvious there are two samples on the middle-left 1010, 1020 that appear to represent duplication in product profiles, so one sample can be dropped.

In the preferred embodiment, the selections and ratings are transferred into the customer's personal data profile. When the customer selects his favorite product (in this case 810), that data point 810 is entered into different parallel databases of data pertaining to the customer's preference. This data can be aggregated by customer outcomes such as amplify, relax/relieve, or sleep based on cannabis chemical profiles.

While this method was illustrated with respect to chai tea, this method could also be used in medicine to help identify ways in which the product is not only better tasting (as in the case of chai) but could also be used to map out products that have a medicinal effect. For example, a higher selection of a particular essential oil as being a preferred choice for customers that want to experience a calming effect may reveal that a higher amount of lavender oil or lavender oil in combination with other components in an essential oil may be strongly correlated with a customer's selecting a particular product as his favorite when identifying a “calming effect” as his primary desired outcome.

Referring now to FIG. 10A, there is shown a flowchart of optimizing personalized product development with the consumer or patient at the center of the process. A consumer or patient 1030 receives one or more sample intake 3D images 1032 created by breeders 1034, growers 1036, dispensaries 1038, or other programs or databases 1040. A seal of approval 1042 can be obtained and applied to the sample intake 3D image(s) 1032.

With physician oversight 1044, a consumer or patient database 1046 is created and maintained, using information from the consumer or patient 1030. Information in the patient database 1046 can be accessed by the consumer or patient via a feedback path 1048. A drug discovery database 1050 organized by patient condition is created from information in the consumer/patient database 1046. A drug standardization and efficacy database 1052 is created and maintained, using information from the drug discovery database 1050 and/or from the consumer or patient 1030 directly. Insurance reimbursement 1054 can be requested from information in the drug standardization and efficacy database 1052, which reimbursement information then updates the consumer/patient database 1046 on an ongoing basis.

Rebranding, distribution, mergers, and/or acquisitions 1056 occur as a result of information stored in the drug standardization and efficacy database 1052. The actions from block 1056 are reflected in updates to the dispensaries 1038, as required.

From information stored in the drug standardization and efficacy database 1052, new formulations 1058 can be created, requiring approval by the Food & Drug Administration (FDA) 1060. If the FDA creates a new category of medications, the consumer or patient 1030 is advised.

The flowchart shown in FIG. 10A can also be modified to relate to products, rather than patients, in which case all steps in the process are suitably revised.

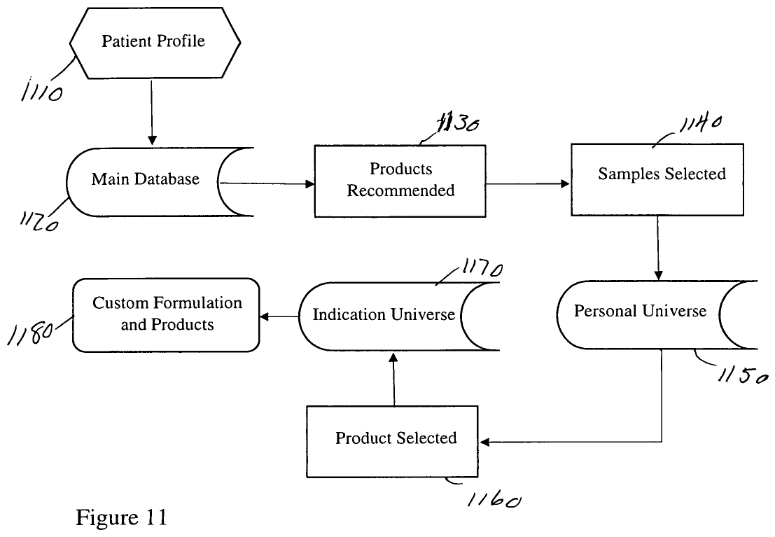

In FIG. 11, a diagram illustrating the components of the current invention is shown. The system comprises a patient/customer profile database 1110, a main database 1120, a list of recommended products 1130, a list of samples selected by the patient/customer 1140, a personal universe dataset 1150 that stores his experiences with prior products and his responses, a listing of recommended products that can be selected by the customer/patient 1160, an indication universe database 1170 that imports data from each customer's respective personal universe and a custom formulations report/database 1180 that can be used to recommend one or more new formulations responsive to data entered into the indication universe database 1170.

In this illustrated system, the customer would use his device (not shown) such as a computer, mobile device, or kiosk to identify themselves to the system. This could be accomplished by commonly known means such as user name/password or could be entered by other means—such as in the case of a kiosk, swiping a credit card or other means for identification. The patient profile 1110 would then be created and could be supplemented with a short questionnaire to help identify his preferences or desired outcomes. For example, in the case of a medicinal product, he may be asked for a condition that he wants to address such as a migraine. All of this collected data is placed into the customer/patient profile database 1110.

The system then directs the customer/patient regions of the cluster diagram containing the maximum range of chemical profiles of the different products in the main database 1120. In the preferred embodiment, the recommended products 1130 would be illustrated, graphically consistent with FIGS. 4-10. The system would then store the selected “dots” or other visualizations representing products in that region that were selected by the customer. A list 1140 of these selected samples would be retained by the system. In the preferred embodiment, the customer would then receive the products corresponding to these dots for trial usage. Following the trial, the customer would once again log into the system using his credentials and enter his preferences in response to his experiences.

The system would store these responses in the personal universe database 1150 based on customer feedback to his experience with the selected sample, for example by using scales of 1 to 10 or a good/bad/indifferent answer construct. This data and the experiences stored by the system in the personal universe could then also be stored in a parallel indication universe database 1170, which would store information regarding the desired outcomes collected during creation of the patent profile 1110 for storage in the indication universe 1170. In sum, the end-to-end experience with the selected products 1160 in combination with the patient profile information 1110 would be retained in the indication universe database 1170 to help improve product recommendations for future patients/customers that indicate that he wants similar outcomes, as discussed in connection with the flowchart shown in FIG. 10A.

The system records each customer experience and overlays underlying product profile data and, if needed, can use his subjective data as well as data similar to them—such as other users with similar conditions or that have had similar subjective profiles—to provide a predicted forecast a direction for them to move in the greatly simplified data universe. These forecasts can be developed using standard multivariable analysis using well-known tools such as MATLAB® or other available software.

In one embodiment, any local “maxima” experience points could be imported into a custom formulations database/report 1180 to help identify the components that appear to be correlated with the most positive customer experiences. In this sense, the system uses the experiential data combined with other data in the personal universe to recommend possible alternative or customer formulations and products 1180. Recombinant products can be made to mimic natural products or those chemicals identified from inhaled smoke streams.

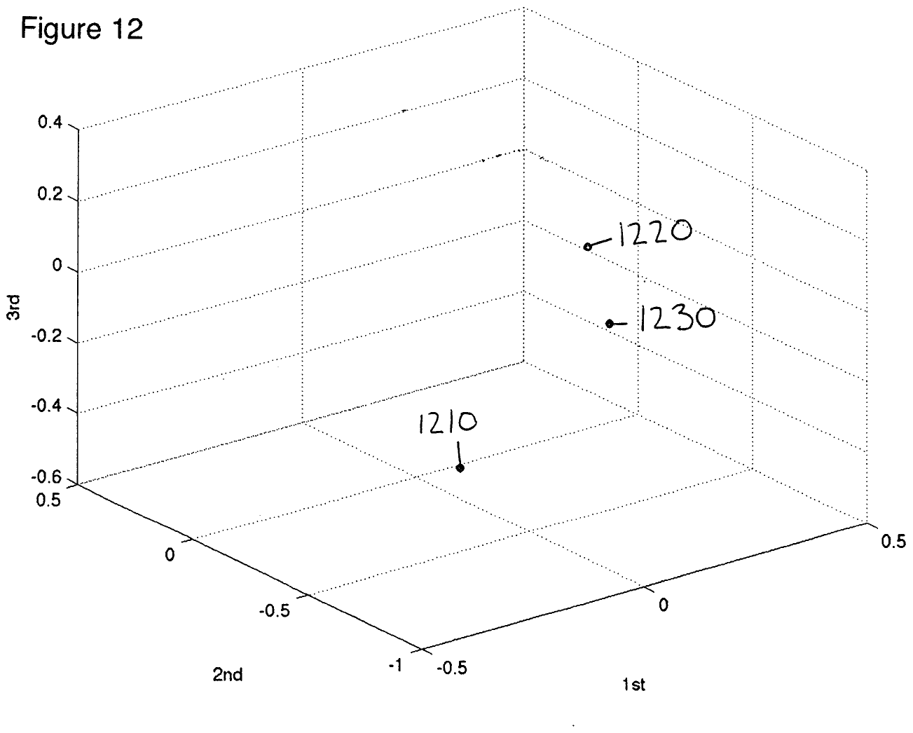

FIG. 12 shows one such possible report that illustrates the product formulations 1210, 1220, 1230 that appear most strongly correlated with the desired customer outcomes. This evolving migraine specific parallel data universe in FIG. 12 will be used to design product that have profiles covering predetermined portions of the patient population and product formulation universe. The geometric location in the product formulation data universe can be tracked directly back to the product characteristics in the original sample using the objective data the original cluster analysis was based upon. New product formulations can be developed to fill holes in the product data universe that show promise.

Each database 1110, 1120, 1130 is updated during this process to link the various inputs to personal experiences for targeted treatments and reproducible personal experiences. Data could also be collected anonymously to help drug discovery scientists and lifestyle coaches to identify useful trends and common benefits across broad customer profiles 1110.

FIG. 13 provides a flowchart of one method of the current invention. A customer/patient begins by creating an account 1310 as explained above using a computer, mobile phone, or other device such as a kiosk. He is then asked by the system what condition he is treating or what sort of effect he is seeking by providing the customer/patient with a view 1320 of the data universe that corresponds with the multivariate statistical de-clustering of data that have been input into the database 1120 by product and is projected on the screen being used by the customer in an intuitive graphic of available products.

An area of the cluster graph is then illuminated or highlighted in the geometric area of correlation with that condition to separate this first group from the rest permitting the user to select 1330 one or more sample products. Following his experience with those products, the customer returns 1340 to his profile on the system and ranks the selected sample products. Assuming he has not found a favorite or otherwise given a product the highest rating, the system would then recommend 1350 additional samples based on the de-clustered data and the preferences/feedback entered by the customer. These steps are repeated as many times as desired in cach instance returning 1360 to his personal profile and getting additional product recommendations 1350 until the customer has selected 1370 a preferred product. Following such selection 1370, the customer can return to the system and check 1380 for new products that may be available that share the characteristics of the selected product. In this way, a customer not only optimizes his selection 1370 of currently available products, but is also given the opportunity by checking 1380 on the system to learn about new products sharing data profiles similar to the selected products.

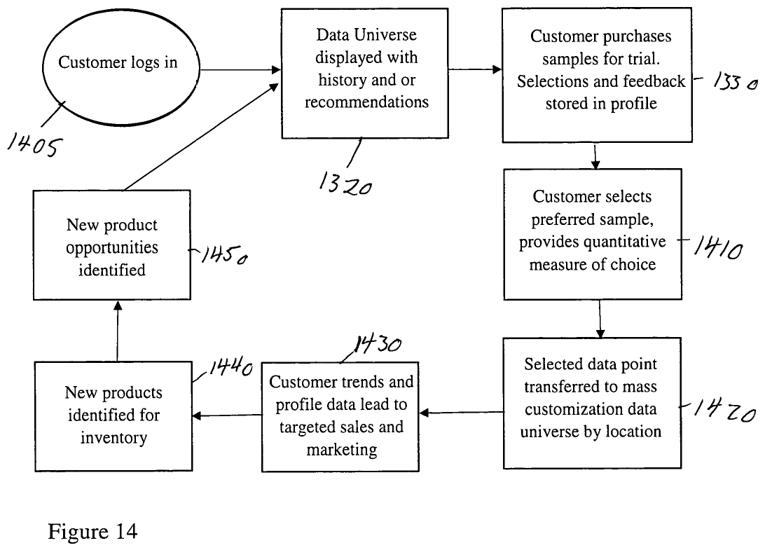

FIG. 14 provides a flowchart of the method outlined above with the additional step(s) of leveraging the system and data collected to help optimize product selection and development. As noted above, in the first step, the customer logs in 1405 to the system and displays the data universe 1320 for the selected product. The customer selects/purchases one or more product samples for trial. When the customer selects the preferred sample 1370, the system further presents a request 1410 to provide a quantitative measure of his choice. The quantitative input received by the system and the associated de-clustered data on the selected product is transferred 1420 to the customization and formulations report/database 1180. The system uses this transferred data to identify 1430 customer trends based in conjunction with the customer/patent profile data 1110 most strongly aligned with the selection of that particular product. In the preferred embodiment, this data is collected anonymously but can be used by product companies and product marketing specialists to help direct targeted sales and marketing campaigns. Furthermore, this data can be used to identify 1440 products that may be beneficial and receive high customer satisfaction based on the collected data. Finally, the system can further use the combination of customer profile data 1110 in conjunction with the product formulation data stored in the main database 1120 and any quantitative data collected 1410 by the system to help identify 1450 new product opportunities or to reach customers that were unable to select 1370 a preferred product. For example, the product data stored in the main database 120 may include unknown peaks by wavelength or mass and will be integrated into the statistical models initially without identification. The effects of each of the inputs can be quantified by covariance related to the measurable outcome(s). Once the compounds in the products, represented by peaks or even unknown masses, are deemed to be important, he is subsequently identified using ppm mass resolution in the old data and the knowledge of the database increases. This method allows for the identification of new compounds of interest and inclusion in the pharmacognosy model as known compounds and ratios in the emerging chemical profile correlation to patient history.

In this way, the customer then interacts with the modeling software to direct them geometrically in the direction of the preferred experience or outcome. In the preferred embodiment, the main database 1120 is updated during this process to link the various inputs to personal experiences for targeted treatments and reproducible personal experiences. Data collected anonymously can further be used to help drug discovery scientists and lifestyle coaches to identify useful trends and common benefits across broad customer and patient profiles.

This method can be applied to development of complex herbal medicines for a variety of indications from tinnitus to treating the common cold. This method can be applied where data are available by factor of juiciest and outcomes can be quantified, even if it is pain management on a scale of 1 to 10.

As new compounds are identified at synergistic levels with the other compounds, a new personalized drug discovery model based on “complex drug discovery” continues to grow, versus the traditional pharmaceutical model of a single compound identified by bioactivity guided fractionation and large clinical trials. This method could also salvage data from earlier clinical trials by harvesting benefits to subsets of the patients revealed in the study while the overall clinical trial may have been deemed a failure or insignificant given the large pool of individuals with varying needs. Helping 20% of the clinical trial population is a potentially huge advancement in patient treatment, not a statistical failure. This system could be used to direct patients to the most effective pain reliever if used over prescription preferences.

This method allows for an evolution of drug discovery into complex drugs, like traditional medicines, based in pharmacognosy that can be tracked and developed using powerful analytical and statistical techniques and related to individuals with personalized medical conditions or needs. This new method of complex drug discovery, called mass customization by statisticians, will essentially provide a scientific basis for what traditional medicine practitioners have relied upon for thousands of years using anecdotal data and personal case studies.

Upon identification of important compounds and specific synergistic potency relationships, new products, processes, and combinations can be developed under carefully controlled conditions to provide these compounds and ratios, or the compounds of interest can be isolated from any source and recombined quantitatively to match the profile of interest identified in the model, RECOMBINATE. Not all migraine customers may respond to the same therapy based on individual body chemistry. However, all resulting treatment populations may share product factors that can be recreated for general use.

This is a major advancement compared to the current, slowly evolving and overly-simplified pharmaceutical trend of “cocktail” dosing strategies combining single drugs into mixtures of two or three single chemicals. Complex drug discovery will likely involve hundreds to thousands of chemicals in traditional medicines while tracking the synergistic ratios of concentrations tied to medical and consumer outcomes. This data collection and application is only possible through the combination of targeted and non-targeted analyses with modern powerful statistical methods such as principal component analysis (PCA).

One promising application appears to be with medical marijuana. Research has shown that the body has hundreds of cannabinoid receptors, binding with different cannabinoids and modulating the body's processes. Experts have discovered that single, isolated cannabinoids are not effective and have gone back to the whole biological plant.

However, sampling the marijuana universe as a patient is nearly impossible as there is no framework by which to compare plants or products. It could take hundreds of samples for a patient to find the right combination, with meaningless strain names causing multiple retries of the same profile without reference. It is already understood that people have different reactions to different plant chemistries. This approach for complex drug discovery based on chemical profile and not strain names is desperately needed.

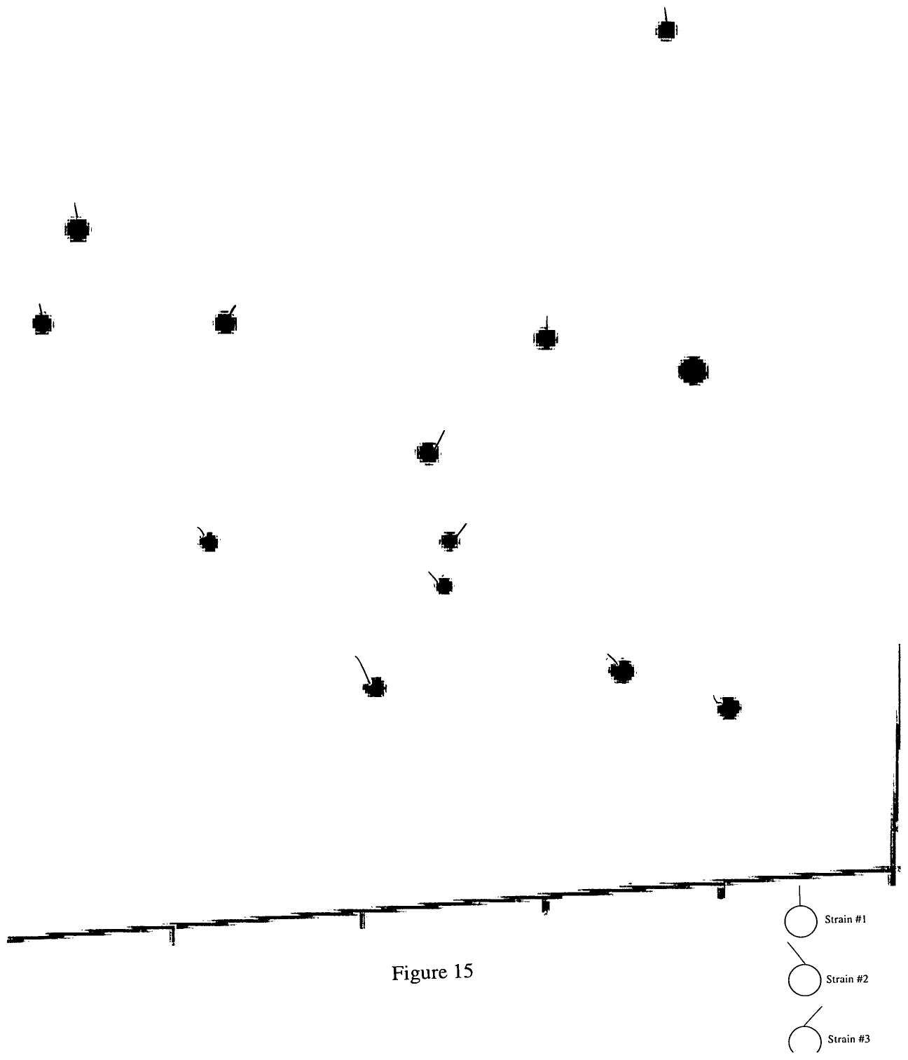

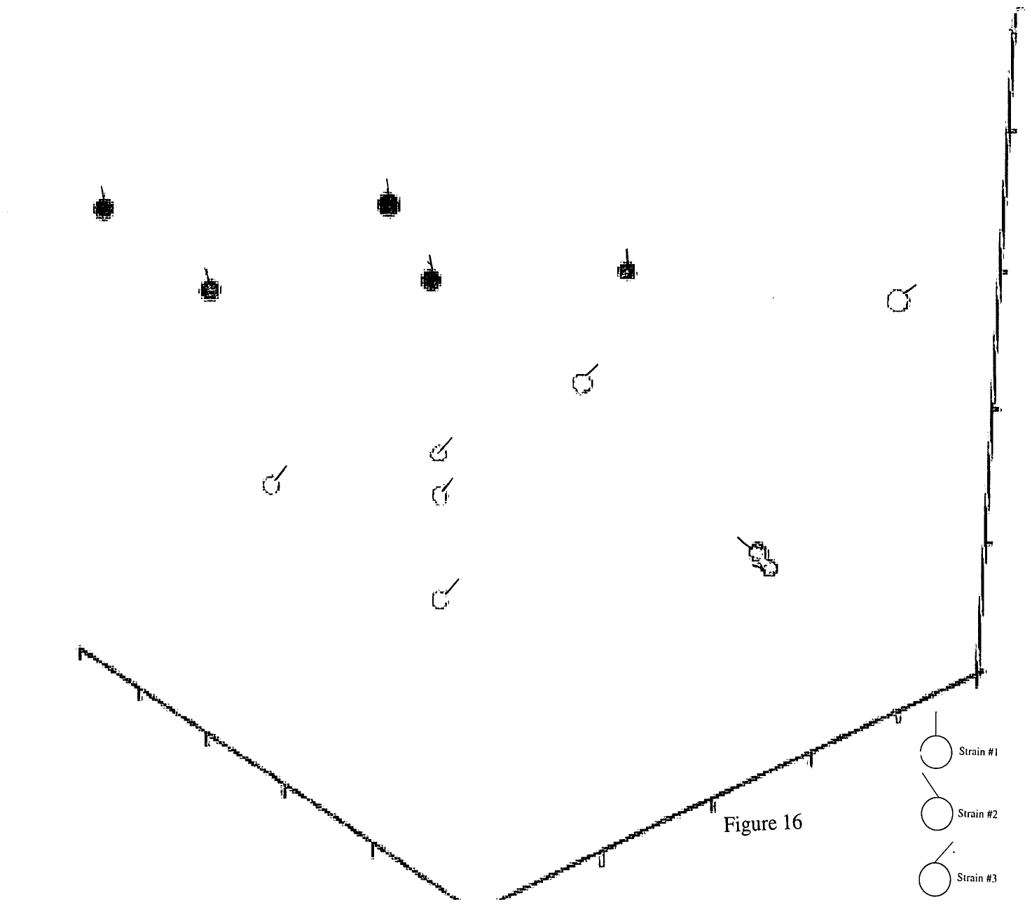

Referring now to FIGS. 15 and 16, there are shown graphs of a sativa indica hybrid chemical profile that unsuccessfully attempts to correlate to strain and of a successful correlation to outcome, respectively. It can be seen that when a system attempts to correlate chemical profile to a strain (FIG. 15), the resulting points are not coherently plotted. However, when the system correlates chemical profile to outcome (FIG. 16), the resulting points are clearly separated and grouped in a useful way.

In another example, a person could track his blood pressure and overall wellbeing versus his diet, lifestyle, and even work environment. The person would identify the factors he feels are important, such as diet, exercise level, and even face time with a contentious coworker to his targeted endpoints, such as blood pressure or attitude on a scale of 1 to 10. Monitoring the various inputs on a quantitative scale from low to high and correlating the data to a targeted outcome may reveal that face time with the boss has more of an effect on blood pressure than the yoga class he started six months ago or increasing the total antioxidants consumed by tracking cumulative daily ORAC values. Seemingly unrelated data can now be combined to monitor the inputs versus the expected outcome.

A patient monitoring his cholesterol could: investigate the effects of different forms and levels of exercise coupled to scientific variations in his diet; or evaluate different dietary supplements versus prescription drugs to realize he is getting synergistic positive effects, and move in that direction or discover that particular combinations are in fact detrimental to his personal health, as evidenced in his changes in cholesterol levels. With enough data, these seemingly confounded contributions can be harvested from the sea of data that one's life generates.

For example, for restless leg syndrome (RLS), one can track blood levels of mg against bioavailability and symptom reduction. Additionally, one can compare mg against Rx drugs for restless leg syndrome to see which is more effective or if synergy exists. For medical marijuana patients, one can cross reference the chemical profile of thousands of different strains vs. results to predict the most effective strains for each patient. Other methods cannot result in these correlations. It is a major advancement to be able to correlate data spanning macro groups and predict and recommend solutions across macro groups into other arenas.

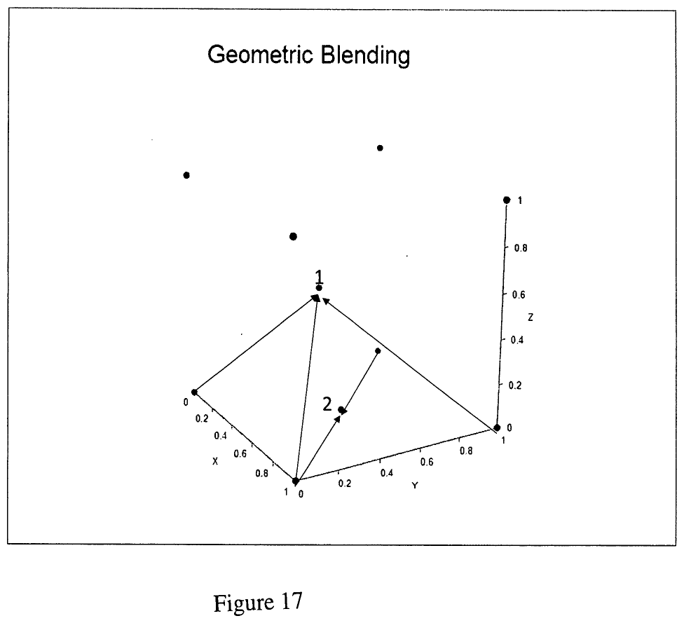

Referring now to FIG. 17, there is shown a display on a point of sale kiosk or vending machine, such as is well known in the field. Vending machine is used to formulate various chemical profile products. In the preferred embodiment, vending machine has eight different compounds or blends as ingredients, each representing a corner of the chemical profile cube generated in FIG. 16. The ingredients can range from single compounds to complex mixtures, and from naturally occurring mixtures to recombinant or synthetic mixtures.

Although a cube is shown as the preferred embodiment in FIG. 17 for descriptive purposes herein, it should be understood that any three-dimensional shape can be used on the display of vending machine. Moreover, any number of ingredients can be used to blend into a predetermined formulation. Naturally occurring complex ingredients can skew the shape of the available formulations shape within the entire mathematical cube. Outlying natural products as close to corners as possible can be chosen as long as linear combinations of the chosen ingredients can produce all the formulations specified within the chemical profile cube.

A customer can choose the desired location on vending machine display representing the desired chemical profile data cube by experience, recommendation, or randomly. Vending machine blends the specified composition from the eight ingredients in the preferred embodiment.

By starting with every corner of the cube, any chemical composition within the cube can be produced mathematically and blended immediately for distribution to the patient or customer. The product designated as number 1 on FIG. 17 is a location within the cube requiring three different ingredients to formulate. Product number 2 is positioned on a face of the cube and requires only two ingredients to formulate. Any location within the box can be made using 2, 3, 6, 8 or more ingredients. When a cartridge, vial, or storage contain vending machine is empty, vending machine can adjust ingredient use away from such cartridge, again as well known in the field of vending machine distribution.

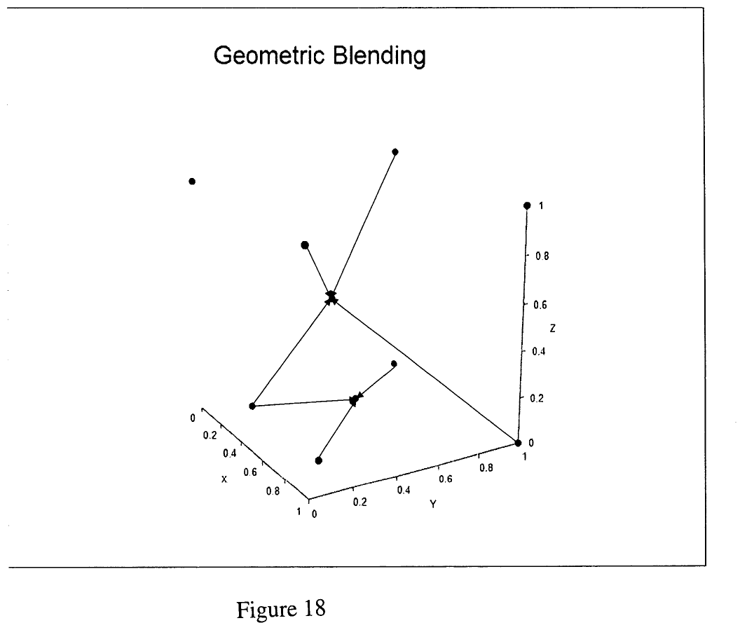

Referring now to FIG. 18, two more sophisticated products are shown on the display of vending machine. In this case, one product is depicted at a location within the cube requiring four different ingredients to formulate and a second product requires three ingredients to formulate.

In sum, the present invention does not merely use data to produce a standardized product, but also enables creation of multiple products to fill the spaces identified by customer preference interaction, making newly targeted products where there are opportunities. By storing data that is both objective as well as subjective, the system provides better and better opportunities to guide individual users to more personalized and subjectively effective compounds, herbs, and treatments.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. A method of optimizing product development based on chemical attribute de-clustering, the steps comprising:
a) analyzing product samples;
b) creating a three-dimensional representation based on factors for client selection;
c) receiving a product selection from a user;
d) receiving preference feedback data from the user including at least one physical attribute or medicinal effect for the product selection; and
e) correlating the preference feedback data to the three-dimensional representation comprising indicia of the preference feedback data to assist the user in product selection.

2. The method of optimizing product selection based on chemical attribute de-clustering in accordance with claim 1, wherein receiving the product selection comprises receiving the product selection on a computing device.

3. The method of optimizing product selection based on chemical attribute de-clustering in accordance with claim 1, wherein the physical or medicinal effects comprise personal feelings for the product selection

4. The method of optimizing product selection based on chemical attribute de-clustering in accordance with claim 1, wherein the physical or medicinal effects comprise a calming effect.

5. The method of optimizing product selection based on chemical attribute de-clustering in accordance with claim 1, wherein the indicia of the preference feedback data comprise at least one geometric location.

6. The method of optimizing product selection based on chemical attribute de-clustering in accordance with claim 1, wherein the indicia of the preference feedback data comprise at least one-color indicia.

7. The method of optimizing product selection based on chemical attribute de-clustering in accordance with claim 1, wherein the indicia of the preference feedback data comprise colored dots.

8. The method of optimizing product selection based on chemical attribute de-clustering in accordance with claim 1, wherein the three-dimensional representation comprises at least one area correlating to the at least one physical or medicinal effect.

9. The method of optimizing product selection based on chemical attribute de-clustering in accordance with claim 1, wherein the product selection comprises at least one item chosen from a group of: food product selection; an herbal product; a drug product; and a cannabis product.

10. A system for optimizing product selection based on chemical attribute de-clustering, comprising:
a) a processor having a non-transitory memory coupled thereto;
b) an input device and an output device coupled to the processor; and wherein the processor is configured to:
i) receive a product selection from a user via the input device;
ii) receive preference feedback data from the user via the input device, comprising at least one physical or medicinal effect for the product selection; and
iii) output the preference feedback data via the output device to generate a three-dimensional representation including indicia of the preference feedback data to assist the user in product selection.

11. The system for optimizing product selection based on chemical attribute de-clustering in accordance with claim 10, wherein the at least one physical or medicinal effect comprises personal feelings for the product selection.

12. The system for optimizing product selection based on chemical attribute de-clustering in accordance with claim 10, wherein the indicia of the preference feedback data comprise colored dots.

13. The system for optimizing product selection based on chemical attribute de-clustering in accordance with claim 13, wherein the three-dimensional representation comprises at least one area correlating to the at least one physical or medicinal effect.

14. The system for optimizing product selection based on chemical attribute de-clustering in accordance with claim 10, wherein the product selection comprises at least one item chosen form a group of: food product selection; an herbal product; a drug product; and a cannabis product.

15. A method of optimizing product development based on a chemical profile database and consumer feedback, the steps comprising:
a) providing information regarding the identity and chemical profile of a plurality of patients;
b) introducing a sample intake 3D image to the information regarding the identity and chemical profile of the plurality of patients;
c) creating a database of information based on the patient chemical profiles;
d) creating a drug discovery database of information organized by patient condition, the drug discovery database being based on the information stored in the database of information based on the patient chemical profiles;
e) creating a drug standardization and efficacy database based on information stored in the drug discovery database and the information regarding the identity and chemical profile of the plurality of patients; and
f) creating a new medicinal formulation based on the information stored in the drug standardization and efficacy database.

16. The method of optimizing product development based on a chemical profile database and consumer feedback in accordance with claim 15, the steps further comprising:
g) providing physician oversight to the database of information based on the patient chemical profiles.

17. The method of optimizing product development based on a chemical profile database and consumer feedback in accordance with claim 15, wherein said sample intake 3D image is derived from at least one of a group of establishments consisting of: i) breeders; ii) growers; iii) dispensaries; and iv) programs.

18. The method of optimizing product development based on a chemical profile database and consumer feedback in accordance with claim 15, wherein the information stored in the database of information based on the patient chemical profiles is introduced to the information regarding the identity and chemical profile of a plurality of patients.

19. The method of optimizing product development based on a chemical profile database and consumer feedback in accordance with claim 15, wherein the Food & Drug Administration (FDA) approves the new medicinal formulation based on information stored in the drug standardization and efficacy database, establishes a new category of medicines, and introduces information to the identity and chemical profile of a plurality of patients.

20. A method of optimizing product development based on a chemical profile database and consumer feedback, the steps comprising:
a) providing information regarding the identity and chemical profile of a plurality of products;
b) introducing a sample intake 3D image to the information regarding the identity and chemical profile of the plurality of products;
c) creating a database of information based on the product chemical profiles;
d) creating a drug discovery database of information organized by product, the drug discovery database being based on the information stored in the database of information based on the product chemical profiles;
e) creating a drug standardization and efficacy database based on information stored in the drug discovery database and the information regarding the identity and chemical profile of the plurality of products; and
f) creating a new medicinal formulation based on the information stored in the drug standardization and efficacy database.

21. The method of optimizing product development based on a chemical profile database and consumer feedback in accordance with claim 20, the steps further comprising:
g) providing physician oversight to the database of information based on the product chemical profiles.

22. The method of optimizing product development based on a chemical profile database and consumer feedback in accordance with claim 20, wherein said sample intake 3D image is derived from at least one of a group of establishments consisting of: i) breeders; ii) growers; iii) dispensaries; and iv) programs.

23. The method of optimizing product development based on a chemical profile database and consumer feedback in accordance with claim 20, wherein the information stored in the database of information based on the product chemical profiles is introduced to the information regarding the identity and chemical profile of a plurality of products.

24. The method of optimizing product development based on a chemical profile database and consumer feedback in accordance with claim 20, wherein the Food & Drug Administration (FDA) approves the new medicinal formulation based on information stored in the drug standardization and efficacy database, establishes a new category of medicines, and introduces information to the identity and chemical profile of a plurality of products.

25. An apparatus for formulating and dispensing a chemical profile product, comprising:
a) a display comprising a plurality of symbols or words representing ingredients;
b) means for selecting a chemical profile product;
c) means for blending at least two ingredients represented on the display to formulate the chemical profile product selected in step (b); and
d) means for dispensing the formulated chemical profile product.

26. The apparatus for formulating and dispensing a chemical profile product in accordance with claim 25, wherein the apparatus is chosen from a group consisting of: a kiosk and a vending machine.

27. The apparatus for formulating and dispensing a chemical profile product in accordance with claim 25, wherein the plurality of symbols or words represent eight ingredients.

28. The apparatus for formulating and dispensing a chemical profile product in accordance with claim 27, wherein the formulated chemical profile product comprises any one of a total number of combinations of at least a portion of the eight ingredients and individual respective dosages thereof.

29. A method of formulating and dispensing a chemical profile product, the steps comprising:
a) displaying a plurality of symbols or words representing ingredients;
b) selecting a chemical profile product;
c) blending at least two ingredients represented on the display to formulate the chemical profile product selected in step (b); and
d) dispensing the formulated chemical profile product.

30. The method of formulating and dispensing a chemical profile product in accordance with claim 29, wherein the method is performed in one of a group consisting of: a kiosk and a vending machine.

31. The method of formulating and dispensing a chemical profile product in accordance with claim 29, wherein the plurality of symbols or words represent eight ingredients.

32. The method of formulating and dispensing a chemical profile product in accordance with claim 31, wherein the formulated chemical profile product comprises any one of a total number of combinations of at least a portion of the eight ingredients and individual respective dosages thereof.
